(19) United States
(12) Patent Application Publication
(10) Pub. No.: US 2018/0335432 A1
(43) Pub. Date: Nov. 22, 2018

(54) System and Method of Detecting Bacterial Infections

(71) Applicants: Arizona Board of Regents acting for and on behalf of Northern Arizona University, Flagstaff, AZ (US); Board of Regents of the Nevada System of Higher Education, on behalf of the University of Nevada, Reno, NV (US)

(72) Inventors: Paul S. Keim, Flagstaff, AZ (US); David P. AuCoin, Reno, NV (US); Erik W. Settles, Flagstaff, AZ (US); Jinhee Yi, Flagstaff, AZ (US)

(21) Appl. No.: 15/983,874

(22) Filed: May 18, 2018

Related U.S. Application Data
(60) Provisional application No. 62/508,307, filed on May 18, 2017.

(51) Int. Cl. G01N 33/569; G01N 33/543

(52) U.S. Cl. CPC G01N 33/56911; G01N 33/54386; G01N 33/54313

(57) ABSTRACT

Implementations of diagnostic systems for detecting a bacterial infection in a subject may include: a device for receiving a sample of a bodily fluid from a subject, a chemical composition configured to react with one or more antibodies in the bodily fluid, and an indicator configured to indicate or respond to a product of the reaction of the chemical composition with one or more antibodies in a bodily fluid received into the device. The antibodies may be produced in response to one or more conserved antigens from one or more bacteria identified as potentially associated with at least one disease associated with the subject. The device may be coated with the chemical composition. The indicator may be configured to communicate to a user of the system a presence or an absence of the one or more antibodies in the bodily fluid.

### CROSS REFERENCE TO RELATED APPLICATIONS

This document claims the benefit of the filing date of U.S. Provisional Patent Application 62/508,307, entitled “System and Method of Detecting Bacterial Infections” to Keim et al. which was filed on May 18, 2017, the disclosure of which is hereby incorporated entirely herein by reference.

### BACKGROUND

### 1. Technical Field

Aspects of this document relate generally to systems for detecting bacterial infections. More specific implementations involve detecting antibodies made by the body of a subject in response to a bacterial or viral infection.

### 2. Background

For infectious diseases, it is still a difficult task for physicians to differentiate viral infections from bacterial infections. Both viral and bacterial diseases trigger similar symptoms including fever, chills and general malaise.

### SUMMARY

Implementations of diagnostic systems for detecting a bacterial infection in a subject may include: a device for receiving a sample of a bodily fluid from a subject, a chemical composition configured to react with one or more antibodies in the bodily fluid, and an indicator configured to indicate or respond to a product of the reaction of the chemical composition with one or more antibodies in a bodily fluid received into the device. The antibodies may be produced in response to one or more conserved antigens from one or more bacteria identified as potentially associated with at least one disease associated with the subject. The device may be coated with the chemical composition. The indicator may be configured to communicate to a user of the system a presence or an absence of the one or more antibodies in the bodily fluid.

Implementations of diagnostic systems for detecting a bacterial infection in a subject may include one, all, or any of the following:

The device may include one of beads or a coated plate.

The subject may be one of a human and an animal.

The bodily fluid may be blood, urine, saliva, or sputum.

A sample of the bodily fluid may be diluted.

The chemical composition may be included in an enzyme-linked immunosorbent assay, a multiplex bead assay, or a peptide microarray.

The system may further include a microprocessor and a memory and a display. The display may be configured to communicate a symbol associated with the indicator.

The indicator generated by the microprocessor and the memory may be a color, a sound, a fluorescence, a symbol, a visually perceptible mark, or a numerical value.

The one or more antibodies may be memory B-cells.

Implementations of diagnostic systems for detecting a bacterial infection in a subject may include: a device for receiving a bodily fluid of a subject. The device may include a chemical composition configured to react with one or more antibodies in the bodily fluid. The antibodies may be produced in response to one or more conserved antigens from one or more bacteria identified associated with at least one disease potentially associated with the subject. The device may include an indicator. The indicator may be configured to indicate or respond to a product of the reaction of the chemical composition with one or more antibodies in a bodily fluid received into the device. The indicator may be configured to communicate to a user of the system the presence or absence of the one or more antibodies.

Implementations of diagnostic systems for detecting a bacterial infection in a subject may include one, all, or any of the following:

The subject may be a human or an animal.

The bodily fluid may be blood, urine, saliva, or sputum.

A sample of the bodily fluid may be diluted.

The device may be a lateral flow assay, an enzyme-linked immunosorbent assay, a multiplex bead assay, or a peptide microarray.

The indicator may be a color, a fluorescence, a symbol, a visually perceptible mark, or a numerical value.

The one or more antibodies may be memory B-cells.

Implementations of a method for detecting a bacterial infection in a subject may include: providing a sample of a bodily fluid; applying the sample of the bodily fluid to a device comprising a chemical composition configured to react with one or more antibodies in the bodily fluid, incubating the sample of bodily fluid for a predetermined amount of time, and interpreting an indicator on the device to determine whether a bacterial infection is present or absent in the subject. The antibodies may be produced in response to one or more conserved antigens from one or more bacteria identified as associated with at least one disease potentially associated with the subject.

Implementations of a method a bacterial infection in a subject may include one, all, or any of the following:

The one or more antibodies may be memory B-cells.

The method may also include diluting the sample of the bodily fluid.

The subject may be a human or an animal.

The foregoing and other aspects, features, and advantages will be apparent to those artisans of ordinary skill in the art from the DESCRIPTION and DRAWINGS, and from the CLAIMS.

### BRIEF DESCRIPTION OF THE DRAWINGS

Implementations will hereinafter be described in conjunction with the appended drawings, where like designations denote like elements, and:

FIGS. 1A, 1B, and 1C are an amino acid sequence comparison of the GroEL protein from different organisms;

FIG. 2 is a pre-infection 2-D Western Blot of B. pseudomallei proteins cross reacting with goat serum;

FIG. 3 is a 2-D Western blot of goat serum 7-day post B. pseudomallei infection cross reacting to B. pseudomallei; and

FIG. 4 is a graph illustrating the results from an enzyme-linked immunosorbent assay (ELISA) used in a particular implementation of a detection method and system.

### DESCRIPTION

This disclosure, its aspects and implementations, are not limited to the specific components, assembly procedures or method elements disclosed herein. Many additional components, assembly procedures and/or method elements known in the art consistent with the intended diagnostic system will become apparent for use with particular implementations from this disclosure. Accordingly, for example, although particular implementations are disclosed, such implementations and implementing components may comprise any shape, size, style, type, model, version, measurement, concentration, material, quantity, method element, step, and/or the like as is known in the art for such diagnostic systems, and implementing components and methods, consistent with the intended operation and methods.

There is a great need for rapid and accurate diagnoses of people that clinically present with diseases in order for medical personnel to respond with the correct therapeutics. For infectious diseases, it is still a difficult task for physicians to differentiate viral infection from bacterial infections. Both viral and bacterial diseases trigger similar symptoms including fever, chills and general malaise. Yet, the therapeutic responses for each are fundamentally different. Specifically, bacterial diseases will respond to antibiotics, while viral diseases do not. Presently, because of the inability to accurately determine the difference between bacterial infections and viral infections, antiviral drugs are misspent on bacterial infections and antibiotics are misspent on viral infections. Diagnostically mistaking a bacterial disease for a viral disease can have severe consequences, including death, when antibiotics are withheld. On the other hand, it is well documented that antibiotics are currently overprescribed for viral diseases and this has had a profound impact on the rise of antibiotic resistant pathogens. Improving the early diagnostics of early bacterial and/or viral infections may have a positive impact on the health of citizens and on the cost of healthcare.

The human and animal immune systems recognize foreign antigens and produces antibodies to inactivate them. Generally, this process takes 1-4 weeks to fully engage but can produce long lasting responses against particular infectious agents. Following an infection, circulating antibodies generated by the body to fight the infection will decrease with time ranging from months to years but memory B-cells that can produce these antibody responses will persist for much longer, at time years to decades. This memory B-cell behavior forms part of the immune protection provided by vaccines or from a subsequent reinfection by the same pathogen. When humans or animals are re-exposed to the same pathogen, the antibody response is very rapid, occurring within days, due to the presence of memory B-cells that quickly multiply and secrete antibodies specific to antigens from that pathogen. Because of this biological principle, reinfection by exactly the same pathogen is uncommon. For some pathogens, there are shared antigens generated by the pathogens in the body, called conserved antigens. Because these conserved antigens are shared by different pathogens, they can, in turn induce cross protection for different infectious diseases. Conserved antigens shared by different pathogens result in the body's formation of cross reacting antibodies. Memory B-cells established by one pathogen can be rapidly, within days, cross stimulated by conserved antigens from a different pathogen.

A system for detecting these conserved antigens could be widely beneficial because all adults and many children have memory B-cells from prior bacterial infections. These bacterial infections can be relatively mild, or severe, and include infections of the sinus, gut, skin, eyes, ears, lungs, and other parts of the body of an organism. During these prior infections, the host immune system responds to defeat the pathogen, with memory B-cells persisting for long time periods. Infection by other bacteria will stimulate a subset of these B-cells within days that recognize and are responding to the conserved antigens to produce antibodies. Accordingly, detecting a rise in a conserved antigen's antibody titer will indicate a bacterial infection even if the patient has never encountered that particular infectious agent before. Viral infections do not induce a similar immune response involving conserved antigens, so no rise in a conserved antigen's antibody titer will be observed during a viral infection. However, it may also be possible to produce a peptide array utilizing conserved viral antigens and then use the peptide array in a diagnostic test to detect a particular one or more possible viruses responsible for the infection. The ability to detect these conserved antigens may decrease diagnostic time and help patients to get well sooner. Utilizing these conserved antigens could also, in various implementations, aid in producing vaccines for a wider range of bacterial infections.

For bacterial pathogens, the foregoing immune response to conserved antigens may be used in implementations of diagnostic methods and diagnostic systems to differentiate a bacterial infection from a viral infection in a patient. In various implementations of diagnostic methods and systems disclosed herein, the proteins that make up conserved bacterial antigens may be used to monitor changes in pathogen induced antibodies in a patient. These selected antigens are then used to capture antibodies from patient serum samples and are used as additional evidence for bacteria-induced disease.

Implementations of a system of detecting bacteria include detecting bacterial infections through the detection of conserved bacterial proteins. Particular implementations also include detecting a class of bacteria based on a patient's health history. Implementations may also include detecting antibodies made by the body of a human or animal in response to a bacterial or viral infection. A diagnostic system for detecting a bacterial infection in a subject may include a device for receiving a sample of a bodily fluid from a subject. The device may be coated with/contain/be coupled with a chemical composition configured to react with one or more antibodies in the bodily fluid. As previously described, the antibodies may be produced in response to one or more conserved antigens from one or more bacteria identified as potentially associated with at least one disease associated with the subject. The antibodies may be memory B-cells. The system may also include an indicator configured to indicate or respond to a product of the reaction of the chemical composition with one or more antibodies in a bodily fluid received into the device. The indicator may be configured to communicate to a user of the system a presence or an absence of the one or more antibodies in the bodily fluid.

In various implementations, the device may include beads such as those used in a multiplex assay. An example of a multiplex assay is assays marketed under the tradename LUMINEX® by Luminex Corporation of Austin, Tex., a Delaware registered company. A multiplex assay may be performed by generating beads that have a specific combination of two fluorophores. By varying the amount of fluorophore on a single bead, a bead may have between 50-200 different florescent bead regions and each bead type can be used in conjugating a specific antigen. After conjugation, the antigenic beads are then mixed and the bead mixture can be incubated with patient serum and each antigen on the beads will react to patient antibodies. The patient antibodies may then be detected with another antibody fluorophore conjugate (secondary antibody). A machine then illuminates and scans for fluorescent signal and this signal indicates the antigen (bead region) and the amount of reactive antibody. Another multiplex technology may be that marketed under the tradename V-PLEX by Meso Scale Discovery of Rockville, Md. In other implementations, the device may include a coat plate as used in enzyme linked immunoassays (ELISA). By non-limiting example, the device may also include glass or plastic chips used as part of a peptide microarray.

The system may also include a microprocessor, a memory, and a display to process the system and generate results for a user. The display of the microprocessor may be configured to communicate a symbol associated with an indicator. In various implementations, the indicator may include a color, a sound, a fluorescence, a symbol, a visually perceptible mark, and a numerical value.

The system may be used in implementations of a method of detecting antibodies produced in response to conserved antigens. Implementations of the method may include providing a sample of a bodily fluid taken from a subject. In various implementations, the bodily fluid may include blood, urine, saliva, sputum, mucus, semen, vaginal fluids, or any bodily fluids known to contain antibodies produced by the immune system of the subject. The method may also include applying the sample of the bodily fluid to a device comprising a chemical composition configured to react with one or more antibodies in the bodily fluid. As previously described the antibodies present in the sample may be from one or more bacteria identified as associated with at least one disease potentially associated with the subject. The diseases potentially associated with the subject may be determined through the health history of the subject or through questions asked during an intake interview. The method may further involve incubating the sample of the bodily fluid for a predetermined amount of time. The predetermined amount of time may be determined by a number of factor such as, by non-limiting example, multiplication of the antibodies, reactivity levels of the chemicals in the device, and other factors that may influence the test. The sample may be incubated at a temperature that is known to be the homeostatic temperature of the subject providing the sample. The method may also include interpreting an indicator on the device to determine whether a bacterial infection is present or absent in the subject. In various implementations, the indicator may be interpreted by an operator of the method or by a machine used in the method.

Various other implementations of diagnostic methods and systems may utilize a number of different methodologies and diagnostic platforms to monitor circulating antibody levels. In some implementations, the methods and systems may be fast and others may be quantitatively precise. In various implementations of a diagnostic system, the system may be able to distinguish between antibody levels present in the patient in the absence of a bacterial infection and those present in the patient during a bacterial infection. Because of the rapid stimulation of memory B-cells due to conserved bacterial antigens from a bacterial infection, it is anticipated that elevated levels of antibodies would occur quickly and using this information, the various diagnostic and method implementations disclosed herein may be able to detect the infection in the early stages of disease.

Particular implementations of a system of detecting bacteria may include a lateral flow assay (LFA) to monitor antibodies' cross reactivity to multiple conserved antigens. In some implementations, these systems may be able to generate a result in just a few minutes of testing. An example of a well-known LFA is an over the counter pregnancy test where, as urine flows through the medium, the reaction by antibodies to human chorionic gonadotropin is used to indicate a negative or positive result to the user by simple visual inspection via a visual marker. Other examples of LFAs that may be employed in various system and method implementations may be any disclosed in U.S. Pat. No. 5,714,341 to Thieme et al, entitled “Saliva assay method and device,” issued Feb. 3, 1998; U.S. Pat. No. 8,470,608 to Babu et al. entitled “Combined visual/fluorescence analyte detection test,” issue date Jun. 25, 2013; U.S Patent Application Publication No. 20050175992 to Aberl et al., entitled “Method for the rapid diagnosis of targets in human body fluids,” published Aug. 11, 2005; U.S Patent Publication No. 2007/0059682 to Aberl et al., entitled “Method to increase specificity and/or accuracy of lateral flow immunoassays,” published Mar. 15, 2007; and German Patent No. DE19622503 to Holger et al, entitled “A method for detecting analytes on a surface,” issued Jul. 9, 1998, the disclosures of each of which are hereby entirely incorporated by reference herein.

Other implementations of a system of detecting bacteria may include an enzyme-linked immunosorbent assay (ELISA). An ELISA test is a plate-based assay technique designed for detecting and quantifying substances such as peptides, proteins, antibodies, and hormones. Other names, such as enzyme immunoassay (EIA), are also used to describe the same technology. Currently ELISA testing is used in the detections of infectious diseases such as Hepatitis B, Hepatitis C, and human immunodeficiency virus (HIV). In still other implementations of a system of detecting bacteria, a peptide array/microarray could be utilized. A peptide microarray is a collection of peptides displayed on a solid surface to study the binding properties and functionality kinetics of protein-protein interactions. Peptide arrays have been used to profile the changing humoral immune responses of individual patients during disease progression. A peptide array may also be used to detect conserved viral antigens for the detection of viral infections. The various testing methods may be utilized to distinguish between viral and bacterial infections and then diagnose a specific infection based on a specific reactive antigen profile. A multiplex bead assay may also be used in order to simultaneously measure multiple analytes or substances in a single test.

In various implementation of diagnostic systems for detecting bacterial infections, antigens may be fixed on a surface and the patient serum may be flowed past the antigens using capillary wicking. For example, a blood drop could be placed on a testing strip at a doctor's office and then flowed past antigens producing a visual effect to allow a doctor to determine whether the presented patient has a bacterial infection (which, if the test was negative, would indicate a non-bacterial infection including viral or fungal infections). In other implementations, other bodily fluids may be used including, by non-limiting example, saliva, sputum, nasal droppings, spinal fluid, lymphatic fluid, semen, vaginal secretions, tears, and any other liquid retrievable from the patient. The particular bodily fluid selected would need to include the antibodies needed for testing. Because of this, any bodily fluid currently known or discovered that contains the antibodies may be used for testing in various system and method implementations. In other systems and methods implementations, a patient's antibodies could be stained with a monoclonal antibody specific to human antibodies and then labeled with a dye. The dye would be designed to allow the doctor to read the result more easily once the bodily fluid has been passed through the diagnostic system implementation (assay). The antibodies may also be first bound to a conserved bacterial antigen before staining.

In implementations of a system of detecting bacterial infection, a doctor or other medical professional could administer the diagnostic system when a patient presents with a fever. The doctor/medical professional may use implementations of a system of detecting bacteria as a first step in the diagnostic process. The system could be used in conjunction with other tests. In some implementations, a primary care doctor may run the test on patients when they are healthy in order to obtain a baseline/control value for comparison when the patient is sick. If the test comes back negative for detecting a bacterial infection, the doctor would be able to shift their attention to a viral infection or other infectious agents. If a positive result for a bacterial infection is obtained, the doctor can prescribe an antibiotic based upon empirical strategies which may be modified, in various system and method implementations, using the results of the test. If the result comes back negative for bacterial infections, antibiotics are contraindicated and doctors will need to perform additional tests to gain additional information on what the cause of the disease is. Where hospitals and other medical practices establish a policy of not prescribing antibiotics in the presence of a negative test from the system and method implementations disclosed herein, the resulting restraint may save the healthcare system a great deal of money, may improve antibiotic stewardship by decreasing drug resistant pathogens, and may do less perturbation/damage to an individual's microbiome.

In various system and method implementations, the system could be used in home setting similar to at-home glucose tests. If the results of the at home test are negative, a user may be able to save themselves a trip to the doctor since the test indicates they are not in need of antibiotics.

Various implementations of a system of detecting bacteria could employ testing method implementations that involve a single conserved antigen, multiple conserved antigens, or various mixtures of conserved antigens. Multiple conserved antigens give the ability to detect more infectious bacteria and may, in various implementations, even provide some diagnostic information concerning the type of bacteria involved in the infection (e.g., gram negative vs. gram positive bacteria).

The various testing system and method implementations disclosed herein monitoring a conserved antigen humoral response for distinguishing bacterial from viral infections, relying upon natural infections to establish memory B-cells. However, other system and method implementations disclosed herein may work in reverse—instead of waiting for previous infections to establish the memory B-cells, the system and method implementations would use one or more conserved antigens via an immunization to immunize children and adults via stimulation of the immune system directly. Immunization with conserved pathogen antigens creates a humoral memory that would be restimulated during a subsequent bacterial infection. In this way, since the memory B-cells have already been prepared to respond to the specific conserved antigen being used in subsequent testing using the principles disclosed herein, this would standardize the patient's body for any subsequent testing and remove the necessity of a prior infection with specific types of bacteria that produce the conserved antigen for the test to work to identify a bacterial infection.

While conventional research has explored differentiation of viral from bacterial infections using polymerase chain reaction (PCR) direct detection this has proven difficult and invariably can only detect a particular pathogen of many possible pathogens. Use of PCR detection is not a general method that will identify a broad range of bacterial infectious agents in a single test.

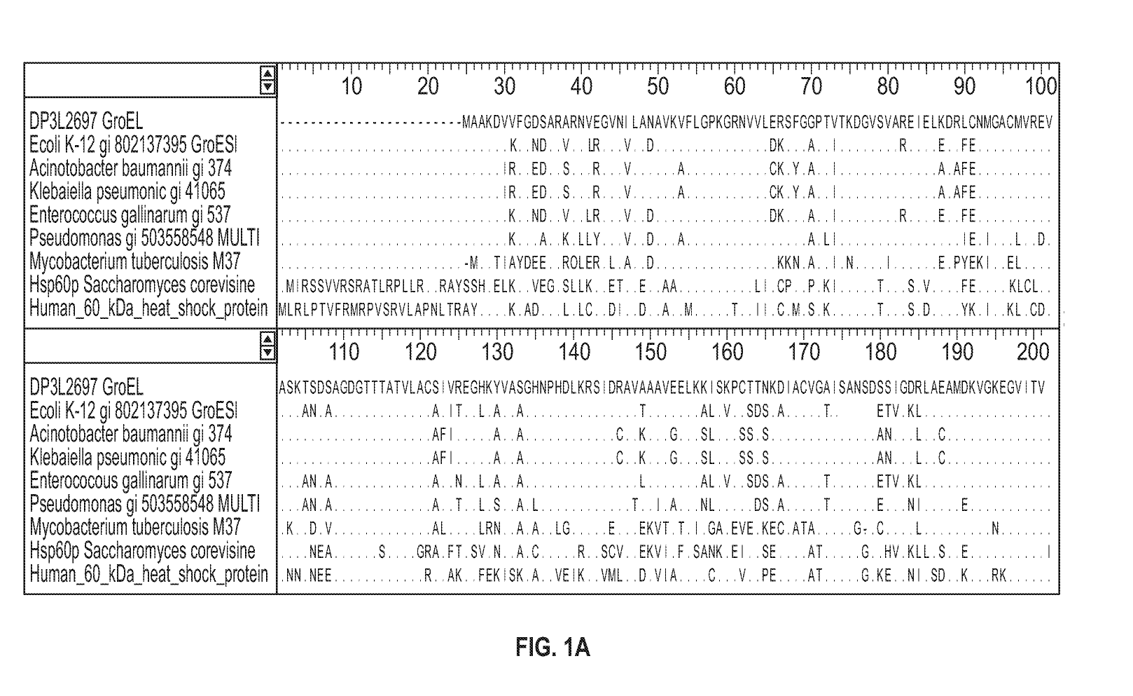

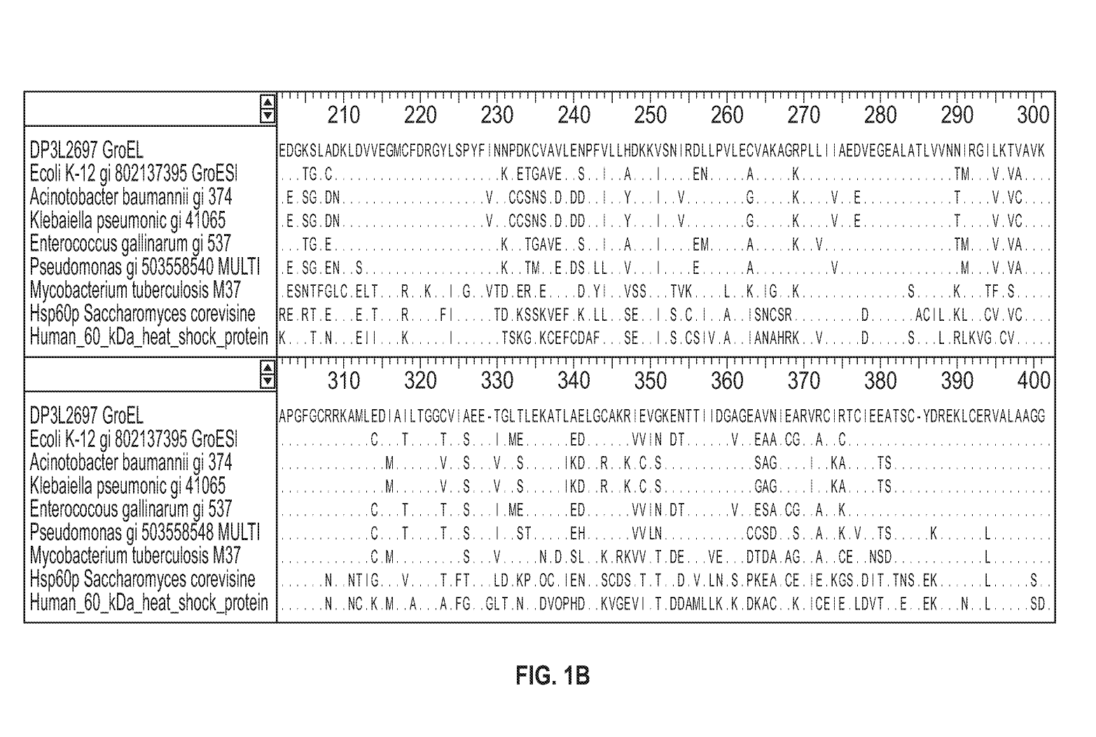

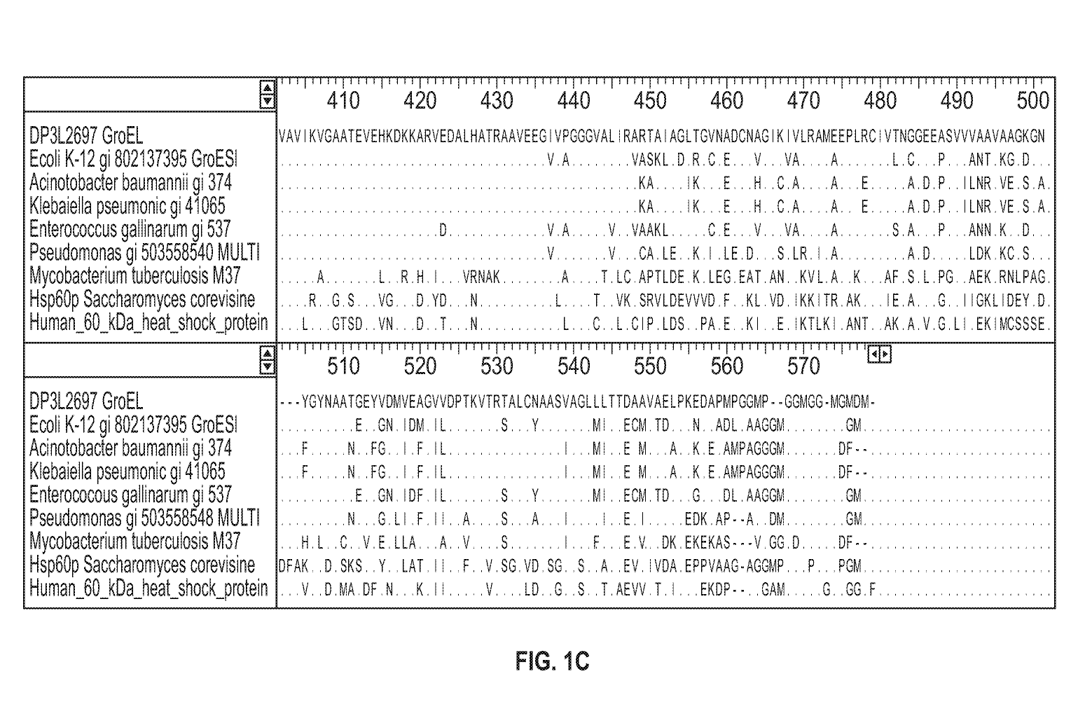

For the exemplary purposes of this disclosure, in a particular system and test implementation, the conserved antigenic proteins from the Gram-negative bacterial pathogens Burkholderia pseudomallei may be used in a test implementation to detect for a bacterial infection. The protein GroEL is found in all bacteria as its function is essential to bacterial cellular function. Its amino acid sequence and three dimensional structure is highly conserved on a wide bacterial taxonomic scale and, accordingly, cross humoral induction of antibodies that respond to this antigenic protein has been observed. Referring to FIGS. 1A-1C, an amino acid sequence comparison of the GroEL protein from different bacteria and mitochondria is illustrated. Highly conserved proteins are observed across pathogenic bacteria and are similar enough to be cross recognized by the adaptive immune system even when the infection is by different pathogens. GroEL is such an example and this figure illustrates its conservation (similarity of chemical structure) between Burkholderia (BPSL2697), E. coli, Acinetobacter, Klebsiella, Enterococcus, Pseudomonas and Mycobacterium which are all potentially human and/or animal pathogens. The amino acid sequence presented in FIGS. 1A-1C of GroEL is from Burkholderia pseudomallei. Other sequences are compared to this reference sequence in FIGS. 1A-1B with dots representing conserved amino acids and the single letter code for variable ones. Humans and yeast have a homologous GroEL protein found in their mitochondria but the GroEL protein is more divergent and less conserved than the bacterial protein.

Various system and method implementations may also utilize antibody cross reactivity to detect bacterial infections. Antibody cross reactivity exists in animals and humans, even if they have never been exposed to a particular pathogen. This is due to previous bacterial infections that generate memory B-cells that secrete cross reacting antibodies. These memory cells are rapidly stimulated by new bacterial infections even thought they were developed for a different bacterial pathogen. Viral infections have distinct antigens and do not generate memory B-cells for bacterial antigens.

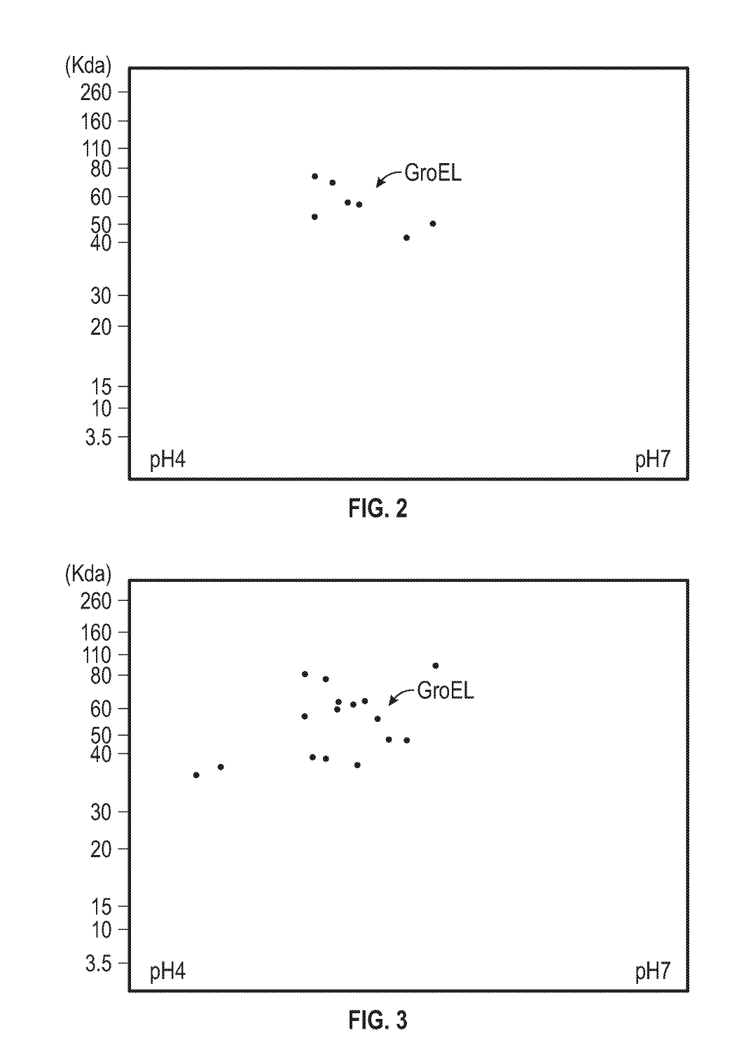

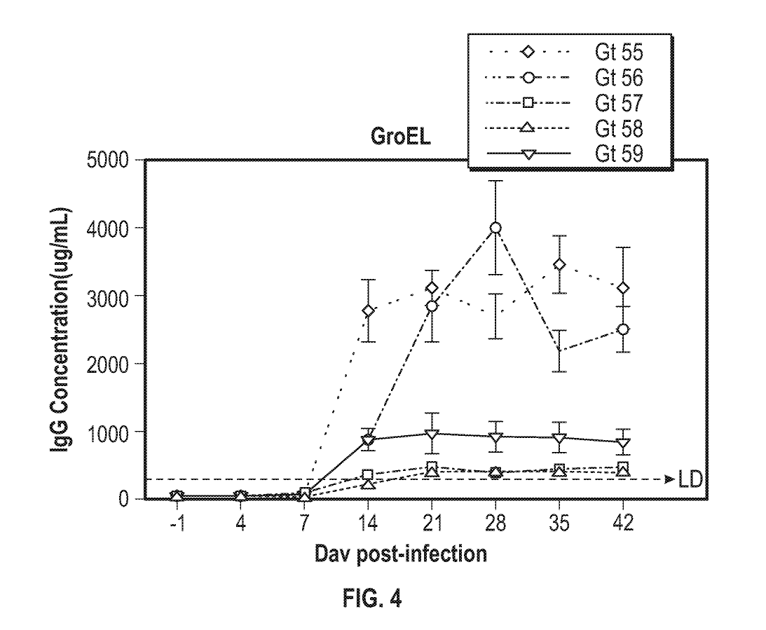

An implementation of a system and method implementation is illustrated in FIGS. 2 and 3. Referring now to FIG. 2, a pre-infection 2-D Western Blot of B. pseudomallei proteins cross reacting with goat serum of pre-challenge is illustrated. The spot on the Western Blot labeled GroEL shows that goat serum is reacting to the presence of this protein, and others as illustrated by the other spots, even though the animal being tested was never been infected with B. pseudomallei. This result demonstrates that anti GroEL cross reacting antibodies are being produced by memory B-cells from a previous but different bacterial infection.

Referring now to FIG. 3, a 7-day post B. pseudomallei infection goat serum cross reacting to B. pseudomallei proteins on a 2-D Western blot result of day 7 post-inoculation with B. pseudomallei is illustrated. This is the same animal whose serum was used in FIG. 2. The result illustrates that the number and intensity of cross reactivity for antigens increases quickly when there are memory B-cells for particular conserved antigens. GroEL and various other cross reacting antigens can be seen on this Western Blot. Previously, detection of this infection would not be possible this early because memory B-cells are not yet developed at 7 days post infection and so the antigens would not be available for detection.

In places where the description above refers to particular implementations of diagnostic systems and implementing components, sub-components, methods and sub-methods, it should be readily apparent that a number of modifications may be made without departing from the spirit thereof and that these implementations, implementing components, sub-components, methods and sub-methods may be applied to other diagnostic systems.

What is claimed is:

1. A diagnostic system for detecting a bacterial infection in a subject, the system comprising:
a device for receiving a sample of a bodily fluid from a subject;
a chemical composition configured to react with one or more antibodies in the bodily fluid, the antibodies produced in response to one or more conserved antigens from one or more bacteria identified as potentially associated with at least one disease associated with the subject; and
an indicator configured to one of indicate and respond to a product of the reaction of the chemical composition with one or more antibodies in a bodily fluid received into the device;
wherein the device is coated with the chemical composition; and
wherein the indicator is configured to communicate to a user of the system one of a presence and an absence of the one or more antibodies in the bodily fluid.

2. The system of claim 1, wherein the device comprises one of beads and a coated plate.

3. The system of claim 1, wherein the subject is one of a human and an animal.

4. The system of claim 1, wherein the bodily fluid is one of blood, urine, saliva, and sputum.

5. The system of claim 1, wherein a sample of the bodily fluid is diluted.

6. The system of claim 1, wherein the chemical composition is comprised in one of an enzyme-linked immunosorbent assay, a multiplex bead assay, and a peptide microarray.

7. The system of claim 1, further comprising a microprocessor and a memory and a display, wherein the display is configured to communicate a symbol associated with the indicator.

8. The system of claim 7, wherein the indicator generated by the microprocessor and the memory is one of a color, a sound, a fluorescence, a symbol, a visually perceptible mark, and a numerical value.

9. The system of claim 1, wherein the one or more antibodies are memory B-cells.

10. A diagnostic system for detecting a bacterial infection in a subject comprising:
a device for receiving a bodily fluid of a subject, the device comprising a chemical composition configured to react with one or more antibodies in the bodily fluid, the antibodies produced in response to one or more conserved antigens from one or more bacteria identified as associated with at least one disease potentially associated with the subject, the device comprising an indicator;
wherein the indicator is configured to one of indicate and respond to a product of the reaction of the chemical composition with one or more antibodies in a bodily fluid received into the device; and
wherein the indicator is configured to communicate to a user of the system one of a presence and an absence of the one or more antibodies.

11. The system of claim 10, wherein the subject is one of a human and an animal.

12. The system of claim 10, wherein the bodily fluid is one of blood, urine, saliva, and sputum.

13. The system of claim 10, wherein a sample of the bodily fluid is diluted.

14. The system of claim 10, wherein the device is one of a lateral flow assay, an enzyme-linked immunosorbent assay, a multiplex bead assay, and a peptide microarray.

15. The system of claim 10, wherein the indicator is one of a color, a fluorescence, a symbol, a visually perceptible mark, and a numerical value.

16. The system of claim 10, wherein the one or more antibodies are memory B-cells.

17. A method for detecting a bacterial infection in a subject, the method comprising:
providing a sample of a bodily fluid;
applying the sample of the bodily fluid to a device comprising a chemical composition configured to react with one or more antibodies in the bodily fluid, the antibodies produced in response to one or more conserved antigens from one or more bacteria identified as associated with at least one disease potentially associated with the subject;
incubating the sample of bodily fluid for a predetermined amount of time; and
interpreting an indicator on the device to determine whether a bacterial infection is one of present and absent in the subject.

18. The method of claim 17, wherein the one or more antibodies are memory B-cells.

19. The method of claim 17, further comprising diluting the sample of the bodily fluid.

20. The method of claim 17, wherein the subject is one of a human and an animal.
